(19) United States
(12) Patent Application Publication
(10) Pub. No.: US 2021/0047605 A1
(43) Pub. Date: Feb. 18, 2021

(54) Control systems and methods for automated clarification of cell culture with high solids content

(71) Applicant: Repligen Corporation, Waltham, MA (US)

(72) Inventors: Derek Carroll, LOS ANGELES, CA (US); Michael Bransby, ALTADENA, CA (US); Philip Yuen, LONG BEACH, CA (US)

(73) Assignee: Repligen Corporation, Waltham, MA (US)

(21) Appl. No.: 16/992,698

(22) Filed: Aug. 13, 2020

Related U.S. Application Data
(60) Provisional application No. 62/886,144, filed on Aug. 13, 2019.

(51) Int. Cl. C12M 1/36; G16B 5/00; B01D 61/22; B01D 61/20; C12M 1/00

(52) U.S. Cl. CPC C12M 41/48; G16B 5/00; C12M 29/04; B01D 61/20; B01D 61/22

(57) ABSTRACT

The present disclosure relates to hollow fiber tangential flow filters, including hollow fiber tangential flow depth filters, for various applications, including bioprocessing applications, systems employing such filters, and methods of filtration using the same.

### CROSS REFERENCE TO RELATED DISCLOSURES

This application claims the benefit of priority under 35 USC § 119(e) to U.S. Provisional application No. 62/886,144 by Derek Carroll, et al., filed Aug. 13, 2019, the entirety of which is incorporated by reference herein for all purposes.

### FIELD OF THE DISCLOSURE

This disclosure relates to the filtration of cell culture fluids using traditional tangential flow filtration and tangential flow depth filtration.

### BACKGROUND

Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing biologicals, using animal or microbial cell culture, filtration is done for clarification, selective removal and concentration of certain constituents from the culture media or to modify the media prior to further processing. Filtration may also be used to enhance productivity by maintaining a culture in perfusion at high cell concentration.

Tangential flow filtration (also referred to as cross-flow filtration or TFF) systems are widely used in the separation of particulates suspended in a liquid phase and have important bioprocessing applications. In contrast to dead-end filtration systems in which a single fluid feed is passed through a filter, tangential flow systems are characterized by fluid feeds that flow across a surface of the filter, resulting in the separation of the feed into two components: a permeate component which has passed through the filter and a retentate component which has not. Compared to dead-end systems, TFF systems are less prone to fouling. Fouling of TFF systems may be reduced further by alternating the direction of the fluid feed across the filtration element as is done in the XCell™ alternating tangential flow (ATF) technology commercialized by Repligen Corporation (Waltham, Mass.), by backwashing the permeate through the filter, and/or by periodic washing.

Modern TFF systems frequently utilize filters comprising one or more tubular filtration elements, such as hollow-fibers or tubular membranes. Where tubular filtration elements are used, they are typically packed together within a larger fluid vessel, and are placed in fluid communication with a feed at one end and at the other end with a vessel or fluid path for the retentate; the permeate flows through pores in the walls of the fibers into the spaces between the fibers and within the larger fluid vessel. Tubular filtration elements provide large and uniform surface areas relative to the feed volumes they can accommodate, and TFF systems utilizing these elements may be scaled easily from development to commercial scale. Despite their advantages, TFF systems filters may foul when filter flux limits are exceeded, and TFF systems have finite process capacities. Efforts to increase process capacities for TFF systems are complicated by the relationship between filter flux and fouling.

Recently, TFF and ATF processes have been engineered utilizing tangential flow depth filters (referred to as tangential flow depth filtration or TFDF) in place of conventional hollow-fiber membranes. Tangential flow depth filters, which combine the reduced fouling behavior associated with tangential flow filtration systems with the increased dirt capacity of depth filtration systems, hold great promise for high density culture and/or continuous filtration applications. This promise, however, may not be realized by simply replacing hollow fiber membrane filters with tangential flow depth filters in existing TFF and ATF processes, and there is an ongoing need for bioprocessing systems and methods that make full use of the benefits of these filters.

### SUMMARY

This disclosure provides new systems and methods for controlling clarification processes in systems that include hollow fiber or TFDF cell retention elements. These systems and methods generally take as user inputs, among other items, the fraction of solids in the culture and the volume of the process vessel (e.g., the bioreactor) used; other inputs such as concentration factor, % yield and permeate volume may be set to default values which can be modified by a user as may be necessary or desirable.

In one aspect, the disclosure relates to filtration methods and/or filtration control methods that comprise receiving one or more of the following values as user inputs into a harvest system: process volume and packed cell volume (PCV); receiving one or more of the following values as administrative inputs into the harvest system: initial concentration factor (CF), permeate throughput volume (PTV), and calculated yield; and running the harvest system in (a) concentration mode, (b) diafiltration mode, and (c) concentration mode. In embodiments according to this aspect, a control algorithm calculates a number of diavolumes processed during diafiltration based on the user and/or administrative inputs. Alternatively, or additionally, one or more of the CF, PTV, yield, process volume and/or packed cell volume etc. may be calculated based on the control algorithm and additional administrative or user inputs (e.g., the number of diavolumes, etc.).

In another aspect, the disclosure relates to methods of automating the harvest of a product from a cell culture, comprising inputting a concentration factor and a permeate throughput volume; starting a run in concentration mode; adding buffer using a diafiltration pump once the input concentration factor has been reached; stopping the diafiltration pump once the calculated or input number of diavolumes have been processed; and ending the run when the total permeate volume has reached the user input or calculated permeate throughput volume.

In yet another aspect, this disclosure relates to a method of performing a filtration process comprising receiving as user inputs: a process volume, a pellet cell volume, a solids cutoff, and optionally a filter retention value; receiving as user or administrative inputs: a percent yield and a permeate throughput volume; calculating run parameters using a control algorithm based on the user and administrative inputs; starting a run in concentration mode; adding buffer using a diafiltration pump based on the run parameters that are calculated; stopping diafiltration once a condition established by a calculated variable or input parameter is achieved (e.g., a certain number of diavolumes have been processed); and ending the run when a condition established by a calculated variable or input parameter is achieved (e.g, the total permeate volume has reached the input or calculated permeate throughput volume).

In various embodiments according to any of the foregoing aspects of this disclosure, the control algorithm uses the percentage of a cell culture that is solid and the percentage of the cell culture which is liquid to calculate an expected product yield. Alternatively, or additionally, the step of performing diafiltration occurs when the system reaches a predetermined percentage of solids, and/or the step of stopping diafiltration further comprises stopping once a calculated percent yield is reached based on the number of diavolumes needed.

The foregoing listing is intended to be exemplary, rather than limiting, and those of skill in the art will recognize that additional aspects and embodiments are presented in the following disclosure.

### BRIEF DESCRIPTION OF THE DRAWINGS

FIG. 1A is a schematic depiction of a TFDF system according to certain embodiments of this disclosure.

FIG. 1B is a schematic depiction of a TFDF system according to certain embodiments of this disclosure.

FIG. 2 is a schematic depiction of a clarification/diafiltration/clarification process according to certain embodiments of this disclosure.

FIG. 3 compares a concentration/diafiltration harvest run and a concentration/diafiltration/concentration harvest run according to certain embodiments of this disclosure

FIG. 4A is a schematic cross-sectional view of a hollow fiber tangential flow depth filter according to the present disclosure;

FIG. 4B is a schematic partial cross-sectional view of three hollow fibers within a tangential flow filter like that shown in FIG. 4A.

FIG. 5 is a schematic cross-sectional view of a wall of a hollow fiber within a tangential flow depth filter like that shown in FIG. 4A.

FIG. 6 is a schematic illustration of a bioreactor system according to the present disclosure.

FIG. 7A is a schematic illustration of a disposable portion of a tangential flow filtering system according to the present disclosure.

FIG. 7B is a schematic illustration of a reusable control system according to the present disclosure.

FIG. 8 is a schematic illustration of a storage medium according to the present disclosure.

FIG. 9 is a schematic illustration of a computing architecture according to the present disclosure.

FIG. 10 is a schematic illustration of a communications architecture according to the present disclosure.

### DETAILED DESCRIPTION

### Overview

The embodiments of this disclosure relate, generally, to TFDF, and in some cases to TFDF systems and methods for use in bioprocessing, particularly in perfusion culture and harvest. One exemplary bioprocessing arrangement compatible with the embodiments of this disclosure includes a process vessel, such as a vessel for culturing cells (e.g., a bioreactor) that produce a desired biological product. This process vessel is fluidly coupled to a TFDF filter housing into which a TFDF filter element is positioned, dividing the housing into at least a first feed/retentate channel and a second permeate or filtrate channel. Fluid flows from the process vessel into the TFDF filter housing are typically driven by a pump, e.g., a mag-lev, peristaltic or diaphragm/piston pump, which may impel fluid in a single direction or may cyclically alternate the direction of flow.

Bioprocessing systems designed to harvest a biological product at the conclusion of a cell culture period generally utilize a large-scale separation device such as a depth filter or a centrifuge in order to remove cultured cells from a fluid (e.g., a culture medium) containing the desired biological product. These large scale devices are chosen in order to capture large quantities of particulate material, including aggregated cells, cellular debris, etc. However, the trend in recent years has been to utilize disposable or single-use equipment in bioprocessing suites to reduce the risks of contamination or damage that that accompanies sterilization of equipment between operations, and the costs of replacing large scale separation devices after each use would be prohibitive.

Additionally, industry trends indicate that bioprocessing operations are being extended or even made continuous. Such operations may extend into days, weeks, or months of operation. Many typical components, such as filters, are unable to adequately perform for such lengths of time without fouling or otherwise needing maintenance or replacement.

Certain systems and methods described herein utilize tubular depth filters, which comprise one or more thick-walled hollow polymer fiber filters. Each hollow fiber is characterized by an inner diameter, an outer diameter and a wall thickness, and is differentiated from standard hollow-fiber membranes by the substantially larger wall thickness and, correspondingly, the larger outer diameter. The larger outer diameters of the thick-walled hollow polymer fibers means that tubular depth filters used in this disclosure may comprise as few as one thick-walled hollow polymer fiber filter, and will generally (but not necessarily) comprise fewer hollow-fibers than corresponding hollow-fiber membrane filters.

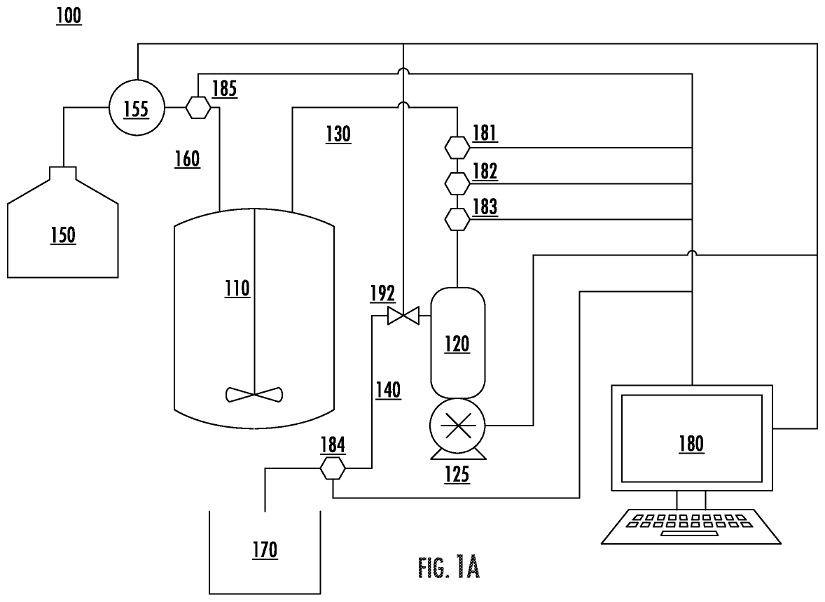

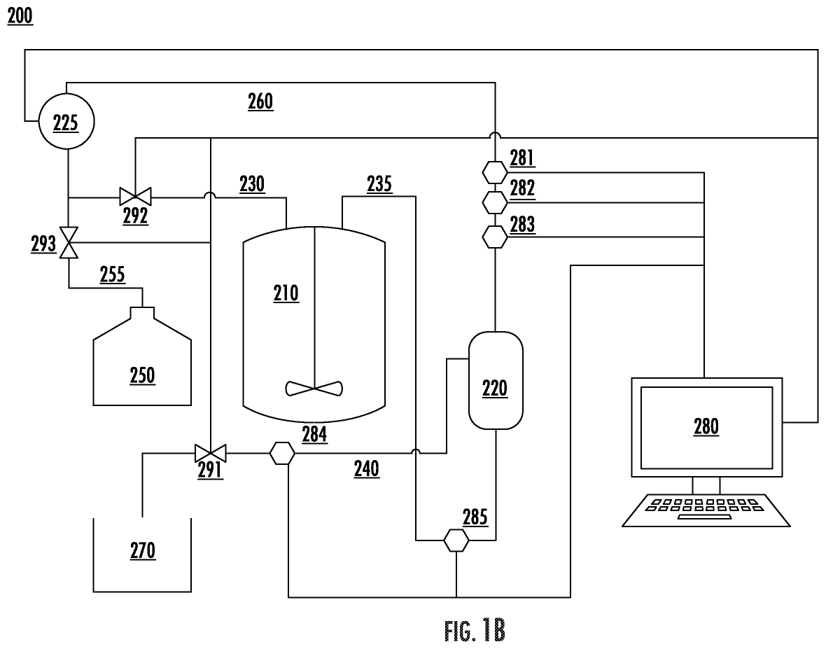

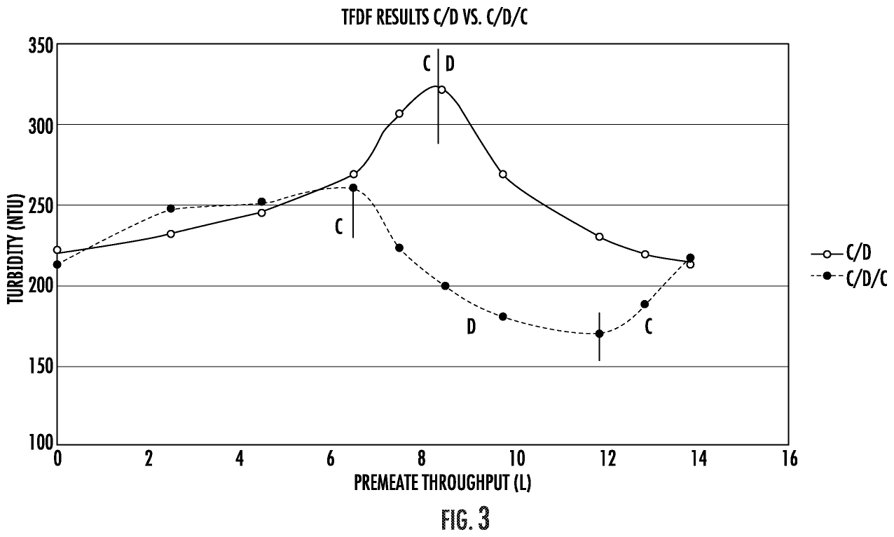

FIGS. 1A and 1B depict exemplary systems for automated clarification of cell cultures used in various embodiments of this disclosure. The automated clarification system 100 depicted in FIG. 1A is configured to provide alternating tangential flow depth filtration and diafiltration. System 100 includes a process vessel 110, such as a bioreactor, and a filter unit 120, which comprises a TFDF filter (not shown) that separates the filter unit into two fluid compartments: a feed/retentate channel 130 and a permeate channel 140 (also referred to filtrate channel). The filter unit 120 is coupled to a positive displacement pump such as a piston or a diaphragm pump as described in, e.g., FIGS. 3c-f of PCT publication No. WO2012026978 to Shevitz, which is incorporated by reference herein. The feed/retentate channel 130 runs between the process vessel 110 and the filter unit 120, while the permeate channel 140 runs to a permeate vessel 170. The system 100 also includes a diafiltration fluid vessel 150. Outflows from the diafiltration fluid vessel 150 pass through a flow control 155 (depicted here as a pump, but which may be a valve or other suitable device), into a diafiltration fluid channel 160 that connects the diafiltration fluid vessel 150 with the process vessel 110.

The system also includes a controller 180, depicted here as a general-purpose computer, but which may be any suitable device that can receive input, send output and perform operations automatically based on pre-programmed instructions (see e.g., FIGS. 8-10). Controller 180 may receive user input through a peripheral device such as a keyboard, touchscreen, etc., and receives process data inputs from one or more sensors 181-183, which measure one or more variables in the culture within one or both of the process vessel 110 and the feed/retentate channel 130. (Though in the figure, the sensors 181-183 are depicted as connected to the feed/retentate channel 130 only). The controller also optionally receives input from one or more sensors 184, 185 in the permeate channel 140 and the diafiltration fluid channel 160, respectively. Variables measured by these sensors can include, without limitation, pressure, flow, pH, temperature, turbidity, optical density, impedance, or other variables relevant to the control of the clarification process.

Based on these inputs, and through execution of a pre-programmed control algorithm or heuristic that implements a control method described in greater detail below, the controller 180 generates one or more outputs, and sends data to components of the system 100 that regulate fluid flows, including the positive displacement pump 125, the diafiltration fluid control 155, and a permeate valve 192 regulating flows through the permeate channel 140.

Turning next to FIG. 1B, an alternative system design utilizes tangential flow filtration and constant-volume diafiltration. System 200 includes a process vessel 210 and a filtration unit 220, but which includes separate outflow 230 and return (retentate) 235 channels, such that the direction of flow through the filtration unit 220 remains constant during operation of the system, rather than alternating as in the system depicted in FIG. 1A. The outflow channel 230 merges with a diafiltration channel 255 from a diafiltration fluid vessel 250 into a single feed channel 260 of the filtration unit 220. The permeate channel 240, permeate vessel 270, controller 280, and sensors 281-285 are substantially as described above for the system depicted in FIG. 1A. Importantly, however, the constant-volume diafiltration process involves the control of multiple fluid channels, and so the controller 280 will send outputs to multiple valves 291, 292, 293, which regulate flows through the permeate channel 240, the process vessel output 230, and the diafiltration fluid output 255, respectively. The controller 280 will also optionally send and/or receive input from a diafiltration pump 225.

It should be noted that certain of the features of the automated systems described above can be modified without modification of other aspects of the system. For instance, although FIG. 1B depicts a TFF system configured for constant-volume diafiltration, those of skill in the art will appreciate that TFF systems may be used which do not provide constant-volume diafiltration.

### Control Algorithms

Clarification is often the first step in a downstream process to recover and purify the product of a cell culture. One of the primary challenges in TFF-based clarification processes is in maximizing product yields (e.g., by maximizing passage of the product into the permeate) while minimizing passage of cells and debris. This is made more complex by the fact that, over time, the fraction of solids in the retentate increases. The concentration process is most efficient when the retentate can concentrate as illustrated by Equation I, below:

$$\% yield = 100 * ( 1 - \frac{1}{C} ) \qquad ( I )$$

where C is the concentration factor

However, at high concentrations of cells and cell debris (i.e., at higher percentages of solids in the retentate), a filter may be more prone to fouling. This increased percentage of solids can be mitigated by running filters in diafiltration mode where the percentage of solids is substantially maintained by the introduction of fresh buffer or media to replace the fluid volume that passes into the permeate. However, running in diafiltration mode for an extended period will also greatly increase the necessary collection volume. The expected yield of a diafiltration process (assuming no retention of the product by the filter) is given by Equation II, below:

% yield=100*(1−e^{-N}) (II)

where N is the number of diavolumes.

Historically, the reduced fouling achieved by lower concentrations of solids has been balanced against the increased collection volume needed for extended diafiltration processes by structuring concentration processes as (a) a first concentration phase, in which the feed/retentate is concentrated to a level that the filter can accommodate without fouling, followed by (b) a diafiltration phase to maximize recovery of the product. However, the inventors have discovered that a process that is structured as (a) a first concentration phase, and (b) a diafiltration phase, followed by (c) a second concentration phase advantageously limits the concentration of solids in the feed/retentate to a threshold that limits the potential for filter fouling, while also reducing the required number of diavolumes.

Certain embodiments of this disclosure relate to a control algorithm for a concentration process which utilizes the starting solid fraction (e.g., expressed as the volume of pelleted cells as a percentage of the volume of input material) (% PCV) and one or more of the following: input material volume (e.g., bioreactor volume) (V), a solid percentage cutoff value (% Solid), and a minimum desired product recovery percentage (% yield). In some embodiments, the algorithm assumes that no product is retained by the filter, though in other embodiments a retention factor or transfer function is used to account for retention of product by the filter and/or the other system components.

Algorithms according to the present disclosure utilize the variable inputs listed above to calculate clarification process variables such as the number of diavolumes to be used in the process, the predicted yield, collection volume, run start and stop times, diafiltration start and stop times, etc. However, in contrast to some currently-used methods, algorithms according to this disclosure can exclude the volume of solids from calculations of these process variables. This approach has several potential advantages, including without limitation (a) ensuring that the concentration factor or the number of diavolumes required is not higher than necessary, and (b) reduction of batch-to-batch variation due to variation in cell content.

As indicated above, algorithms according to some embodiments of this disclosure calculate the concentration factor and other process variables so that the fraction of solids is kept below a predetermined threshold solid concentration or % Solid during the run. This can be achieved, for example, by starting a diafiltration pump when a solid concentration is detected that is at or above the % Solid threshold. The diafiltration pump will run, and the diafiltration step will continue, until a number of diavolumes required to achieve the % Yield are delivered. Then, the diafiltration pump stops and the system continues to run in concentration mode until the concentration factor is reached.

In some embodiments, this disclosure relates to a method of harvesting a product from a cell culture. The harvest process is defined by a control algorithm that takes 5 (or optionally 6) inputs such as the following:
V=Process/Bioreactor Volume
PCV=% of Pellet Cell Volume
% Solid=the maximum percent of the cell culture that will be solid phase material prior to terminating concentration mode
Yield=% of product in the bioreactor that will be recovered (assuming ideal passage through the filter and the rest of the system)
R=optional retention factor to correct calculations for any product retained by the filter or the rest of the system. Can be set to zero to assume ideal filtration.
Permeate Throughput Volume=the volume of the permeate pool.

A process according to these embodiments will begin in concentration mode, with the diafiltration pump off so that the retentate is concentrated in the bioreactor. The % PCV is compared to the % Solid and the process switches from concentration mode to diafiltration mode based the remaining bioreactor volume and the permeate throughput values calculated according to equations III and IV below:

$$\begin{matrix}Remaining Bioreactor Volume = \frac{PCV * V}{\% Solid} & ( III ) \\ Permeate Throughput Volume = V * ( 1 - \frac{PCV}{\% Solid} ) & ( IV )\end{matrix}$$

As discussed above, after the process switches from the initial concentration mode to diafiltration mode, it will continue in diafiltration mode until the number of diavolumes required to achieve the desired % Yield have been passed, calculated according to equation V below.

$$\# of Diavolumes = \frac{ln \frac{( PCV - 1 ) * Solids * ( 1 - Yield )}{PCV * ( Solids + R - 1 ) - Solids * R}}{R - 1} \qquad ( V )$$

It will be clear to those of skill in the art that methods according to this group of embodiments may be particularly well suited to implementation in systems that include sensors or other means for monitoring the number of diavolumes added to the system and/or and the volume of permeate passed by the filter. The total volume of buffer added to the system during the diafiltration mode is calculated according to the following equations:

Buffer Volume=Diavolume*# of Diavolumes (VI)

Total Volume=Buffer Volume+Concentration Permeate Throughput Volume (VII)

Embodiments of this disclosure can be used with a variety of tangential flow filtration systems configured with a diafiltration pump and diafiltration fluid source, including without limitation TFF systems utilizing thin-walled hollow fibers and TFF systems using thick-walled hollow fibers, which are described in greater detail below.

Diafiltration fluids used in embodiments of this disclosure include any suitable fluid used in the art. For instance, in many cases fresh cell culture media is used (e.g., to minimize stress or insult to cells), while in other cases saline solutions (e.g., phosphate buffered saline, Tris-buffered saline, etc.) may be used. Other aqueous media can also be used, including without limitation water, in which case the rate at which the diafiltration fluid is added to the system (and consequently the process time) is optionally adjusted to reduce or minimize any shock to cells due to exposure to a solution that is not osmotically balanced.

In some instances, the solids content, or pelleted cell volume, of a culture are used in generating control algorithm outputs. The solids content of a culture or solution is not necessarily fixed, however, and can be manipulated in certain embodiments of this disclosure, e.g., by flocculation, which may increase the total solids content and/or the mean particle size and may consequently reduce the potential for membrane fouling during a process. Thus, in some embodiments a solids content that is modified before or during the filtration run by, e.g., flocculation is used as an input variable.

### TFDF

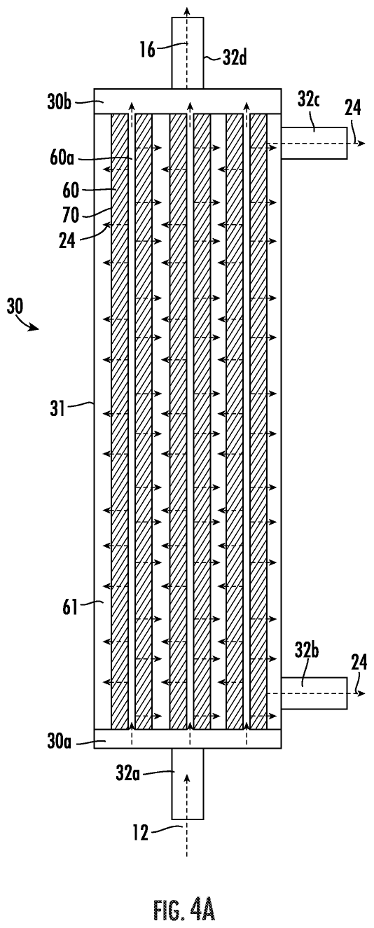

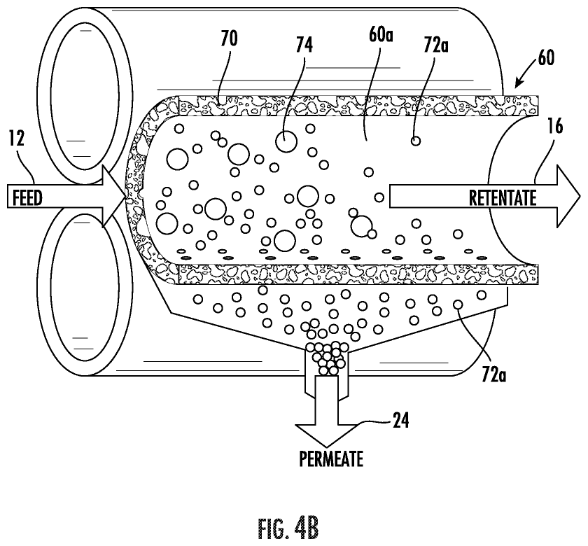

A schematic cross-sectional view of a thick-walled hollow fiber tangential flow filter 30 in accordance with present disclosure is shown in FIG. 4A. The hollow fiber tangential flow filter 30 includes parallel hollow fibers 60 extending between an inlet chamber 30a and an outlet chamber 30b. A fluid inlet port 32a provides a flow 12 to the inlet chamber 30a and a retentate fluid outlet port 32d receives a retentate flow 16 from the outlet chamber 30b. The hollow fibers 60 receive the flow 12 through the inlet chamber 30a. The flow 12 is introduced into a hollow fiber interior 60a of each of the hollow fibers 60, and a permeate flow 24 passes through walls 70 of the hollow fibers 60 into a permeate chamber 61 within a filter housing 31. The permeate flow 24 travels to permeate fluid outlet ports 32b and 32c. Although two permeate fluid outlet ports 32b and 32c are employed to remove permeate flow 24 in FIG. 4A, in other embodiments, only a single permeate fluid outlet port may be employed. Filtered retentate flow 16 moves from the hollow fibers 60 into the outlet chamber 30b and is released from the hollow fiber tangential flow filter 30 through retentate fluid outlet port 32d.

FIG. 4B is a schematic partial cross-sectional view of three hollow fibers 60 within a hollow fiber tangential flow filter analogous to that shown in FIG. 4A, and shows the separation of an inlet flow 12 (also referred to as a feed) which contains large particles 74 and small particles 72a into a permeate flow 24 containing a portion of the small particles and a retentate flow 16 containing the large particles 74 and a portion of the small particles 72a that does not pass through the walls 70 of the follow fibers 60.

Tangential flow filters in accordance with the present disclosure include tangential flow filters having pore sizes and depths that are suitable for excluding large particles (e.g., cells, micro-carriers, or other large particles), trapping intermediate-sized particles (e.g., cell debris, or other intermediate-sized particles), and allowing small particles (e.g., soluble and insoluble cell metabolites and other products produced by cells including expressed proteins, viruses, virus like particles (VLPs), exosomes, lipids, DNA, or other small particles). As used herein a “microcarrier” is a particulate support allowing for the growth of adherent cells in bioreactors.

Tangential flow depth filters in accordance with various embodiments of the present disclosure do not have a precisely defined pore structure. Particles that are larger than the “pore size” of the filter will be stopped at the surface of the filter. A significant quantity of intermediate-sized particles, on the other hand, enter the wall for the filter, and are entrapped within the wall before emerging from the opposing surface of the wall. Smaller particles and soluble materials can pass though the filter material in the permeate flow. Being of thicker construction and higher porosity than many other filters in the art, the filters can exhibit enhanced flow rates and what is known in the filtration art as “dirt loading capacity,” which is the quantity of particulate matter a filter can trap and hold before a maximum allowable back pressure is reached.

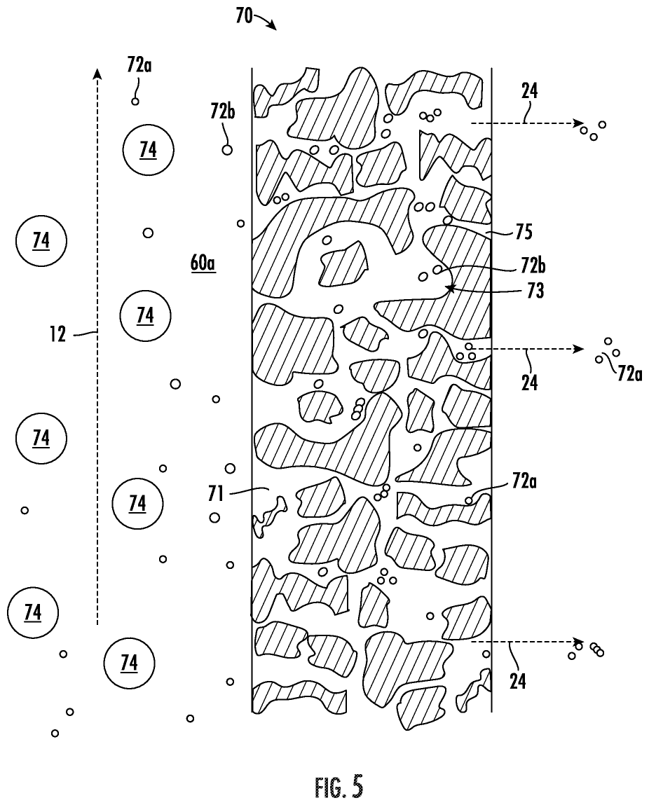

In this regard, FIG. 5 is a schematic cross-sectional illustration of a wall 70 of a hollow fiber 60 used in conjunction with a hollow fiber tangential flow filter 30 like that of FIG. 4A. In FIG. 5, a flow 12 comprising large particles 74, small particles 72a, and intermediate-sized particles 72b is introduced into the fluid inlet port 32a of the hollow fiber tangential flow filter 30. The large particles 74 pass along the inner surface of the wall 70 that forms the hollow fiber interior 60a (also referred to herein as the fiber lumen) of the hollow fibers and are ultimately released in the retentate flow. The wall 70 includes tortuous paths 71 that capture certain elements (i.e., intermediate-sized particles 72b) of the flow 12 as a portion of the flow 12 passes through the wall 70 of hollow fiber tangential flow filter 30 while allowing other particles (i.e., small particles 72a) to pass through the wall 70 as part of the permeate flow 24. In the schematic cross-sectional illustration of FIG. 5, settling zones 73 and narrowing channels 75 are illustrated as capturing intermediate-size particles 72b which enter the tortuous paths 71, while allowing smaller particles 72a to pass through the wall 70, thus trapping intermediate-size particles 72b and causing a separation of the intermediate-size particles 72b from smaller particles 72a in the permeate flow 24. This method is thus different from filtering obtained by the surface of standard thin wall hollow fiber tangential flow filter membranes, wherein intermediate-size particles 72b can build up at the inner surface of the wall 70, clogging entrances to the tortuous paths 71.

In this regard, one of the most problematic areas for various filtration processes, including filtration of cell culture fluids such as those filtered in perfusion and harvest of cell culture fluids, is decreased mass transfer of target molecules or particles due to filter fouling. The present disclosure overcomes many of these hurdles by combining the advantages of tangential flow filtration with the advantages of depth filtration. As in standard thin wall hollow fiber filters using tangential flow filtration, cells are pumped through the lumens of the hollow fibers, sweeping them along the surface of the inner surface of the hollow fibers, allowing them to be recycled for further production. However, instead of the protein and cell debris forming a fouling gel layer at the inner surface of the hollow fibers, the wall adds what is referred to herein as a “depth filtration” feature that traps the cell debris inside the wall structure, enabling increased volumetric throughput while maintaining close to 100% passage of typical target proteins in various embodiments of the disclosure. Such filters may be referred to herein as tangential flow depth filters.

As illustrated schematically in FIG. 5, tangential flow depth filters in accordance with various embodiments of the present disclosure do not have a precisely defined pore structure. Particles that are larger than the “pore size” of the filter will be stopped at the surface of the filter. A significant quantity of intermediate-sized particles, on the other hand, enter the wall for the filter, and are entrapped within the wall before emerging from the opposing surface of the wall. Smaller particles and soluble materials can pass though the filter material in the permeate flow. Being of thicker construction and higher porosity than many other filters in the art, the filters can exhibit enhanced flow rates and what is known in the filtration art as “dirt loading capacity,” which is the quantity of particulate matter a filter can trap and hold before a maximum allowable back pressure is reached.

Despite a lack of a precisely defined pore structure, the pore size of a given filter can be objectively determined via a widely used method of pore size detection known as the “bubble point test.” The bubble point test is based on the fact that, for a given fluid and pore size, with constant wetting, the pressure required to force an air bubble through a pore is inversely proportional to the pore diameter. In practice, this means that the largest pore size of a filter can be established by wetting the filter material with a fluid and measuring the pressure at which a continuous stream of bubbles is first seen downstream of the wetted filter under gas pressure. The point at which a first stream of bubbles emerges from the filter material is a reflection of the largest pore(s) in the filter material, with the relationship between pressure and pore size being based on Poiseuille's law which can be simplified to P=K/d, where P is the gas pressure at the time of emergence of the stream of bubbles, K is an empirical constant dependent on the filter material, and d is pore diameter. In this regard, pore sizes determined experimentally herein are measured using a POROLUX™ 1000 Porometer (Porometer NV, Belgium), based on a pressure scan method (where increasing pressure and the resulting gas flow are measured continuously during a test), which provides data that can be used to obtain information on the first bubble point size (FBP), mean flow pore size (MFP) (also referred to herein as “mean pore size”), and smallest pore size (SP). These parameters are well known in the capillary flow porometry art.

In various embodiments, hollow fibers for use in the present disclosure may have, for example, a mean pore size ranging from 0.1 microns (μm) or less to 30 microns or more, typically ranging from 0.2 to 5 microns, among other possible values.

In various embodiments, the hollow fibers for use in the present disclosure may have, for example, a wall thickness ranging from 1 mm to 10 mm, typically ranging from 2 mm to 7 mm, more typically about 5.0 mm, among other values.

In various embodiments, hollow fibers for use in the present disclosure may have, for example, an inside diameter (i.e., a lumen diameter) ranging from 0.75 mm to 13 mm, ranging from 1 mm to 5 mm, 0.75 mm to 5 mm, 4.6 mm, among other values. In general, a decrease in inside diameter will result in an increase in shear rate. Without wishing to be bound by theory, it is believed that an increase in shear rate will enhance flushing of cells and cell debris from the walls of the hollow fibers.

Hollow fibers for use in the present disclosure may have a wide range of lengths. In some embodiments, the hollow fibers may have a length ranging, for example, from 200 mm to 2000 mm in length, among other values.

The hollow fibers for use in the present disclosure may be formed from a variety of materials using a variety of processes.

For example, hollow fibers may be formed by assembling numerous particles, filaments, or a combination of particles and filaments into a tubular shape. The pore size and distribution of hollow fibers formed from particles and/or filaments will depend on the size and distribution of the particles and/or filaments that are assembled to form the hollow fibers. The pore size and distribution of hollow fibers formed from filaments will also depend on the density of the filaments that are assembled to form the hollow fibers. For example, mean pore sizes ranging from 0.5 microns to 50 microns may be created by varying filament density.

Suitable particles and/or filaments for use in the present disclosure include both inorganic and organic particles and/or filaments. In some embodiments, the particles and/or filaments may be mono-component particles and/or mono-component filaments. In some embodiments, the particles and/or filaments may be multi-component (e.g., bi-component, tri-component, etc.) particles and/or filaments. For example, bi-component particles and/or filaments having a core formed of a first component and a coating or sheath formed of a second component, may be employed, among many other possibilities.

In various embodiments, the particles and/or filaments may be made from polymers. For example, the particles and/or filaments may be polymeric mono-component particles and/or filaments formed from a single polymer, or they may be polymeric multi-component (i.e., bi-component, tri-component, etc.) particles and/or filaments formed from two, three, or more polymers. A variety of polymers may be used to form mono-component and multi-component particles and/or filaments including polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others.

In various embodiments, a porous wall of a filter may have a density that is a percentage of volume that the filaments take up compared to an equivalent solid volume of the polymer. For example, a percent density may be calculated by dividing the mass of the porous wall of the filter by the volume that the porous wall takes up and comparing the result, in ratio form, to the mass of a non-porous wall of the filament material divided by the same volume. A filter having a specific density percentage may be produced during manufacturing that has a direct relation to the amount of variable cell density (VCD) at which the filter can operate without fouling. A density of a porous wall of a filter may additionally or alternatively be expressed by a mass per volume (e.g., grams/cm3).

Table 1 below shows exemplary data of six filters having a density percentage of about 51%. Although the second filter P3 of FIG. 6 and the filters of Table 1 below have a pore size of about 4 μm and a density percentage of about 51%, other filters are contemplated having a different pore size and density percentage, e.g., a filter having a density percentage of about 53% and a pore size of about 2 μm with a 90% nominal retention.

TABLE 1
Parameters for Filters Having a Pore Size of About 4 μm
Scale (sn B651486632) Caliper (SN 11344515)

| Sample | Weight (g) | Length (in) | OD (cm) max | OD (cm) min | Avg | ID (cm) | Density |
|---|---|---|---|---|---|---|---|
| 1 | 10.7 | 27.3 | 0.63246 | 0.62992 | 0.63119 | 0.15 | 0.522931121 |
| 2 | 13 | 33.46 | 0.64262 | 0.63246 | 0.63754 | 0.15 | 0.507494299 |
| 3 | 13 | 33.42 | 0.6477 | 0.63246 | 0.64008 | 0.15 | 0.503843298 |
| 4 | 5.8 | 14.88 | 0.64008 | 0.63246 | 0.63627 | 0.15 | 0.511296131 |
| 5 | 5.8 | 14.88 | 0.63754 | 0.62992 | 0.63373 | 0.15 | 0.515646644 |
| 6 | 5.9 | 14.88 | 0.635 | 0.63246 | 0.63373 | 0.15 | 0.524537103 |
| | | | | | | Avg | 0.514291433 |
| | | | | | | StDev | 0.00831614 |

Particles may be formed into tubular shapes by using, for example, tubular molds. Once formed in a tubular shape, particles may be bonded together using any suitable process. For instance, particles may be bonded together by heating the particles to a point where the particles partially melt and become bonded together at various contact points (a process known as sintering), optionally, while also compressing the particles. As another example, the particles may be bonded together by using a suitable adhesive to bond the particles to one another at various contact points, optionally, while also compressing the particles. For example, a hollow fiber having a wall analogous to the wall 70 that is shown schematically in FIG. 5 may be formed by assembling numerous irregular particles into a tubular shape and bonding the particles together by heating the particles while compressing the particles.

Filament-based fabrication techniques that can be used to form tubular shapes include, for example, simultaneous extrusion (e.g., melt-extrusion, solvent-based extrusion, etc.) from multiple extrusion dies, or electrospinning or electrospraying onto a rod-shaped substrate (which is subsequently removed), among others.

Filaments may be bonded together using any suitable process. For instance, filaments may be bonded together by heating the filaments to a point where the filaments partially melt and become bonded together at various contact points, optionally, while also compressing the filaments. As another example, filaments may be bonded together by using a suitable adhesive to bond the filaments to one another at various contact points, optionally while also compressing the filaments.

In particular embodiments, numerous fine extruded filaments may be bonded together to at various points to form a hollow fiber, for example, by forming a tubular shape from the extruded filaments and heating the filaments to bond the filaments together, among other possibilities.

In some instances, the extruded filaments may be melt-blown filaments. As used herein, the term “melt-blown” refers to the use of a gas stream at an exit of a filament extrusion die to attenuate or thin out the filaments while they are in their molten state. Melt-blown filaments are described, for example, in U.S. Pat. No. 5,607,766 to Berger. In various embodiments, mono- or bi-component filaments are attenuated as they exit an extrusion die using known melt-blowing techniques to produce a collection of filaments. The collection of filaments may then be bonded together in the form of a hollow fiber.

In certain beneficial embodiments, hollow fibers may be formed by combining bicomponent filaments having a sheath of first material which is bondable at a lower temperature than the melting point of the core material. For example, hollow fibers may be formed by combining bicomponent extrusion technology with melt-blown attenuation to produce a web of entangled biocomponent filaments, and then shaping and heating the web (e.g., in an oven or using a heated fluid such as steam or heated air) to bond the filaments at their points of contact. An example of a sheath-core melt-blown die is schematically illustrated in U.S. Pat. No. 5,607,766 in which a molten sheath-forming polymer and a molten core-forming polymer are fed into the die and extruded from the same. The molten bicomponent sheath-core filaments are extruded into a high velocity air stream, which attenuates the filaments, enabling the production of fine bicomponent filaments. U.S. Pat. No. 3,095,343 to Berger shows an apparatus for gathering and heat-treating a multi-filament web to form a continuous tubular body (e.g., a hollow fiber) of filaments randomly oriented primarily in a longitudinal direction, in which the body of filaments are, as a whole, longitudinally aligned and are, in the aggregate, in a parallel orientation, but which have short portions running more or less at random in non-parallel diverging and converging directions. In this way, a web of sheath-core bicomponent filaments may be pulled into a confined area (e.g., using a tapered nozzle having a central passageway forming member) where it is gathered into tubular rod shape and heated (or otherwise cured) to bond the filaments.

In certain embodiments, as-formed hollow fiber may be further coated with a suitable coating material (e.g., PVDF) either on the inside or outside of the fiber, which coating process may also act to reduce the pore size of the hollow fiber, if desired.

Hollow fibers such as those described above may be used to construct tangential flow filters for bioprocessing and pharmaceutical applications. Examples of bioprocessing applications in which such tangential flow filters may be employed include those where cell culture fluid is processed to separating cells from smaller particles such as proteins, viruses, virus like particles (VLPs), exosomes, lipids, DNA and other metabolites.

Such applications include perfusion applications in which smaller particles are continuously removed from cell culture medium as a permeate fluid while cells are retained in a retentate fluid returned to a bioreactor (and in which equivalent volumes of media are typically simultaneously added to the bioreactor to maintain overall reactor volume). Such applications further include clarification or harvest applications in which smaller particles (typically biological products) are more rapidly removed from cell culture medium as a permeate fluid.

Hollow fibers such as those described above may be used to construct tangential flow depth filters for particle fractionation, concentration and washing. Examples of applications in which such tangential flow filters may be employed include the removal of small particles from larger particles using such tangential flow depth filters, the concentration of microparticles using such tangential flow depth filters and washing microparticles using such tangential flow filters.

A specific example of a bioreactor system 10 for use in conjunction with the present disclosure will now be described. With reference to FIGS. 3, 4A and 4B, 6, 7A and 7B the bioreactor system 10 includes a bioreactor vessel 11 containing bioreactor fluid 13, a tangential flow filtering system 14, and a control system 20. The tangential flow filtering system 14 is connected between a bioreactor outlet 11a and bioreactor inlet 11b to receive bioreactor fluid 12 (also referred to as a bioreactor feed), which contains, for example, cells, cell debris, cell metabolites including waste metabolites, expressed proteins, etc., through bioreactor tubing 15 from the bioreactor 11 and to return a filtered flow 16 (also referred to as a retentate flow or bioreactor return) through return tubing 17 to the bioreactor 11. The bioreactor system 10 cycles bioreactor fluid through the tangential flow filtering system 14 which removes various materials (e.g., cell debris, soluble and insoluble cell metabolites and other products produced by cells including expressed proteins, viruses, virus like particles (VLPs), exosomes, lipids, DNA, or other small particles) from the bioreactor fluid and returns cells to allow the reaction in the bioreactor vessel 11 to continue. Removing waste metabolites allows the continued proliferation of cells within the bioreactor, thereby allowing the cells to continue to express recombinant proteins, antibodies or other biological materials that are of interest.

The bioreactor tubing 15 may be connected, for example, to the lowest point or dip tube of the bioreactor 11 and the return tubing 17 may be connected to the bioreactor 11, for example, in the upper portion of the bioreactor volume and submerged in the bioreactor fluid 13.

The bioreactor system 10 includes an assembly comprising a hollow fiber tangential flow filter 30 (described in more detail above), a pump 26, and associated fittings and connections. Any suitable pump may be used in conjunction with the present disclosure including, for example, peristaltic pumps, positive displacement pumps, and pumps with levitating rotors inside the pumpheads, among others. As a specific example, the pump 26 may include a low shear, gamma-radiation stable, disposable, levitating pumphead 26a, for example, a model number PURALEV® 200SU low shear re-circulation pump manufactured by Levitronix, Waltham, Mass., USA. The PURALEV® 200SU includes a magnetically levitated rotor inside a disposable pumphead, and stator windings in the pump body, allowing simple removal and replacement of the pumphead 26a.

The flow of bioreactor fluid 12 passes from the bioreactor vessel 11 to the tangential flow filtering system 14 and the return flow of the bioreactor fluid 16 passes from the tangential flow filtering system 14 back to the bioreactor vessel 11. A permeate flow 24 (e.g., containing soluble and insoluble cell metabolites and other products produced by cells including expressed proteins, viruses, virus like particles (VLPs), exosomes, lipids, DNA, or other small particles) is stripped from the flow of bioreactor material 12 by the tangential flow filtering system 14 and carried away from the tangential flow filtering system 14 by tubing 19. The permeate flow 24 is drawn from the hollow fiber tangential flow system 14 by a permeate pump 22 into a storage container 23.

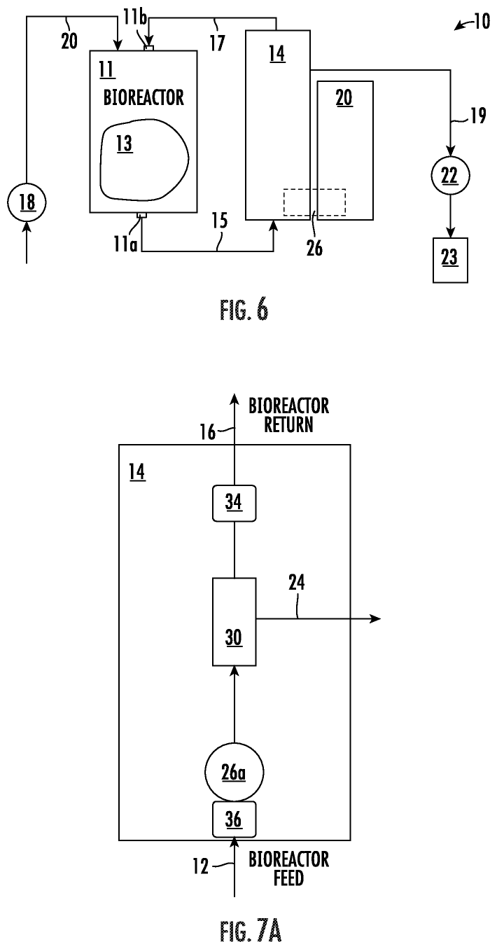

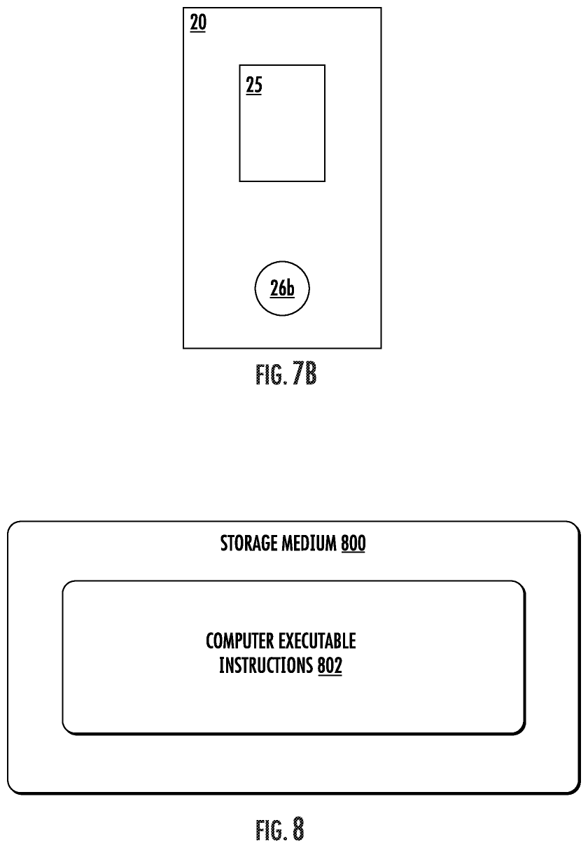

In the embodiment shown, the tangential flow filtering system 14 (see FIG. 7A) includes a disposable pumphead 26a, which simplifies initial set up and maintenance. The pumphead 26a circulates the bioreactor fluid 12 through the hollow fiber tangential flow filter 30 and back to the bioreactor vessel 11. A non-invasive transmembrane pressure control valve 34 may be provided in line with the flow 16 from the hollow fiber tangential flow filter 30 to the bioreactor vessel 11, to control the pressure within the hollow fiber tangential flow filter 30. For example, the valve 34 may be a non-invasive valve, which resides outside tubing carrying the return flow 16 that squeezes the tubing to restrict and control the flow, allowing the valve to regulate the applied pressure on the membrane. Alternatively, or in addition, a flow controller 36 may be provided at the pumphead 26a inlet in order to provide pulsed flow to the hollow fiber tangential flow filter 30, as described in more detail below. The permeate flow 24 may be continually removed from the bioreactor fluid 13 which flows through the hollow fiber tangential flow filter 30. The pumphead 26a and the permeate pump 22 are controlled by the control system 20 to maintain the desired flow characteristics through the hollow fiber tangential flow filter 30.

The pumphead 26a and hollow fiber tangential flow filter 30 in the tangential flow filtering system 14 may be connected by flexible tubing allowing easy changing of the elements. Such tubing allows aseptic replacement of the hollow fiber tangential flow filter 30 in the event that the hollow fiber tangential flow filter 30 becomes plugged with material and therefore provides easy exchange to a new hollow fiber assembly.

The tangential flow filtering system 14 may be sterilized, for example, using gamma irradiation, ebeam irradiation, or ETO gas treatment.

Referring again to FIG. 4, during operation, two permeate fluid outlet ports 32b and 32c may be employed to remove permeate flow 24 in some embodiments. In other embodiments, only a single permeate fluid outlet port may be employed. For example, permeate flow 24 may be collected from the upper permeate port 32c only (e.g., by closing permeate port 32b) or may be collected from the lower permeate port 32b only (e.g., by draining the permeate flow 24 from the lower permeate port 32b while the permeate port 32c closed or kept open). In certain beneficial embodiments, the permeate flow 24 may be drained from the lower permeate port 32b to reduce or eliminate Sterling flow, which is a phenomenon where an upstream (lower) end of the of the hollow fibers 60 (the high-pressure end) generates permeate that back-flushes the downstream (upper) end of the hollow fibers 60 (the low-pressure end). Draining the permeate flow 24 from the lower permeate port 32b leaves air in contact with the upper end of the of the hollow fibers 60 minimizing or eliminating Sterling flow.

In certain embodiments, the bioreactor fluid 12 may be introduced into the hollow fiber tangential flow filter 30 at a constant flow rate.

In certain embodiments, the bioreactor fluid may be introduced into the hollow fiber tangential flow filter 30 in a pulsatile fashion (i.e., under pulsed flow conditions), which has been shown to increase permeate rate and volumetric throughput capacity. As used herein “pulsed flow” is a flow regime in which the flow rate of a fluid being pumped (e.g., fluid entering the hollow fiber tangential flow filter) is periodically pulsed (i.e., the flow has periodic peaks and troughs). In some embodiments, the flow rate may be pulsed at a frequency ranging from 1 cycle per minute or less to 2000 cycles per minute or more (e.g., ranging from 1 to 2 to 5 to 10 to 20 to 50 to 100 to 200 to 500 to 1000 to 2000 cycles per minute) (i.e., ranging between any two of the preceding values). In some embodiments, the flow rate associated with the troughs is less than 90% of the flow rate associated with the peaks, less than 75% of the flow rate associated with the peaks, less than 50% of the flow rate associated with the peaks, less than 25% of the flow rate associated with the peaks, less than 10% of the flow rate associated with the peaks, less than 5% of the flow rate associated with the peaks, or even less than less than 1% of the flow rate associated with the peaks, including zero flow and periods of backflow between the pulses.

Pulsed flow may be generated by any suitable method. In some embodiments, pulsed flow may be generated using a pump such as a peristaltic pump that inherently produces pulsed flow. For example, tests have been run by applicant which show that switching from a pump with a magnetically levitated rotor like that described above under constant flow conditions to a peristaltic pump (which provides a pulse rate of about 200 cycles per minute) increases the amount of time that a tangential flow depth filter can be operated before fouling (and thus increases the quantity of permeate that can be collected).

In some embodiments, pulsed flow may be generated using pumps that otherwise provide a constant or essentially constant output (e.g., a positive displacement pump, centrifugal pumps including magnetically levitating pump, etc.) by employing a suitable flow controller to control the flow rate. Examples of such flow controllers include those having electrically controlled actuators (e.g. a servo valve or solenoid valve), pneumatically controlled actuators or hydraulically controlled actuators to periodically restrict fluid entering or exiting the pump. For example, in certain embodiments, a flow controller 36 may be placed upstream (e.g., at the inlet) or downstream (e.g., at the outlet) of a pump 26 like that described hereinabove (e.g., upstream of pumphead 26a in FIG. 7A) and controlled by a controller 20 to provide pulsatile flow having the desired flow characteristics.

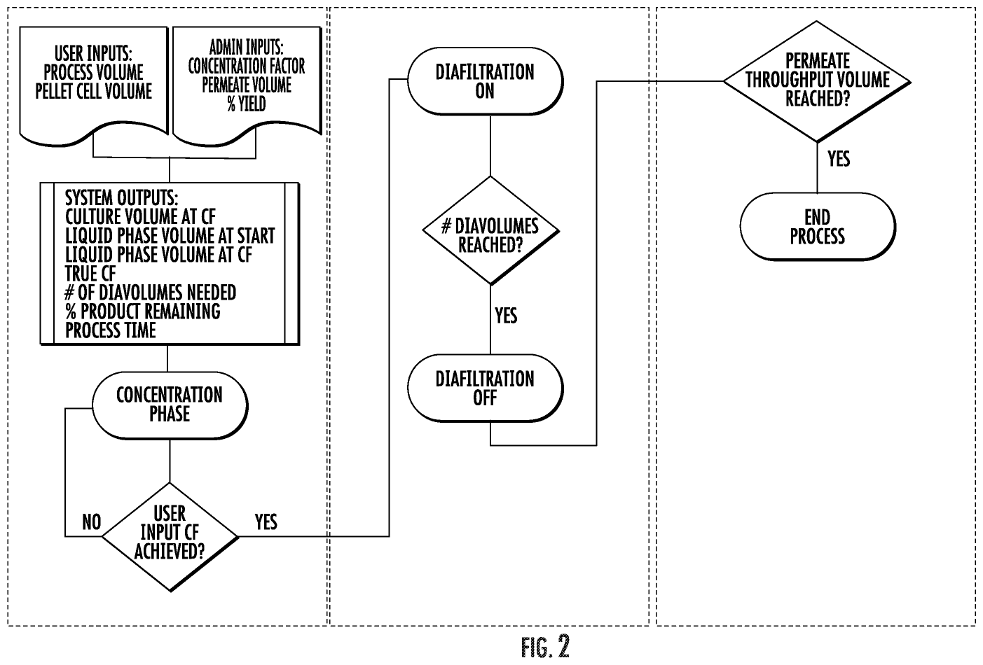

FIG. 8 illustrates an embodiment of a storage medium 800. Storage medium 800 may comprise any non-transitory computer-readable storage medium or machine-readable storage medium, such as an optical, magnetic or semiconductor storage medium. In various embodiments, storage medium 800 may comprise an article of manufacture. In some embodiments, storage medium 800 may store computer-executable instructions 802, such as computer-executable instructions to implement one or more of logic flows, processes, techniques, or operations disclosed hereby (e.g., the clarification/diafiltration/clarification process of FIG. 2). Examples of a computer-readable storage medium or machine-readable storage medium may include any tangible media capable of storing electronic data, including volatile memory or non-volatile memory, removable or non-removable memory, erasable or non-erasable memory, writeable or re-writeable memory, and so forth. Examples of computer-executable instructions may include any suitable type of code, such as source code, compiled code, interpreted code, executable code, static code, dynamic code, object-oriented code, visual code, and the like. The embodiments are not limited in this context.

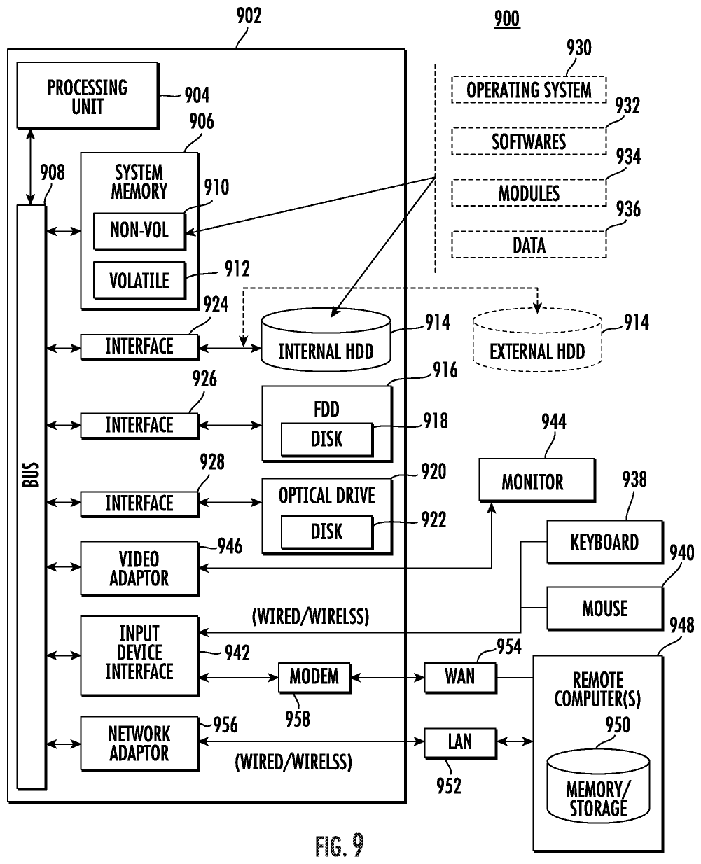

FIG. 9 illustrates an embodiment of an exemplary computing architecture 900 that may be suitable for implementing various embodiments as previously described. In various embodiments, the computing architecture 900 may comprise or be implemented as part of an electronic device. In some embodiments, the computing architecture 900 may be representative, for example, of one or more component described herein. In some embodiments, computing architecture 900 may be representative, for example, of a computing device that implements or utilizes one or more portions of components and/or techniques disclosed herein, such as one or more of controller 180, sensors 181-185, flow control 155, valve 192, controller 280, sensors 281-285, valves 291, 292, 293, and the control algorithms. The embodiments are not limited in this context.

As used in this application, the terms “system” and “component” and “module” may refer to a computer-related entity, either hardware, a combination of hardware and software, software, or software in execution, examples of which are provided by the exemplary computing architecture 900. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 900 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 900.

As shown in FIG. 9, the computing architecture 900 comprises a processing unit 904, a system memory 906 and a system bus 908. The processing unit 904 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 904.

The system bus 908 provides an interface for system components including, but not limited to, the system memory 906 to the processing unit 904. The system bus 908 can be any of several types of bus structure that may further interconnect to a memory bus (with or without a memory controller), a peripheral bus, and a local bus using any of a variety of commercially available bus architectures. Interface adapters may connect to the system bus 908 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 906 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory (e.g., one or more flash arrays), polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 9, the system memory 906 can include non-volatile memory 910 and/or volatile memory 912. In some embodiments, system memory 906 may include main memory. A basic input/output system (BIOS) can be stored in the non-volatile memory 910.

The computer 902 may include various types of computer-readable storage media in the form of one or more lower speed memory units, including an internal (or external) hard disk drive (HDD) 914, a magnetic floppy disk drive (FDD) 916 to read from or write to a removable magnetic disk 918, and an optical disk drive 920 to read from or write to a removable optical disk 922 (e.g., a CD-ROM or DVD). The HDD 914, FDD 916 and optical disk drive 920 can be connected to the system bus 908 by an HDD interface 924, an FDD interface 926 and an optical drive interface 928, respectively. The HDD interface 924 for external drive implementations can include at least one or both of Universal Serial Bus (USB) and Institute of Electrical and Electronics Engineers (IEEE) 994 interface technologies. In various embodiments, these types of memory may not be included in main memory or system memory.

The drives and associated computer-readable media provide volatile and/or nonvolatile storage of data, data structures, computer-executable instructions, and so forth. For example, a number of program modules can be stored in the drives and memory units 910, 912, including an operating system 930, one or more application programs 932, other program modules 934, and program data 936. In one embodiment, the one or more application programs 932, other program modules 934, and program data 936 can include or implement, for example, the various techniques, applications, and/or components described herein.

A user can enter commands and information into the computer 902 through one or more wire/wireless input devices, for example, a keyboard 938 and a pointing device, such as a mouse 940. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 904 through an input device interface 942 that is coupled to the system bus 908 but can be connected by other interfaces such as a parallel port, IEEE 1394 serial port, a game port, a USB port, an IR interface, and so forth.

A monitor 944 or other type of display device is also connected to the system bus 908 via an interface, such as a video adaptor 946. The monitor 944 may be internal or external to the computer 902. In addition to the monitor 944, a computer typically includes other peripheral output devices, such as speakers, printers, and so forth.

The computer 902 may operate in a networked environment using logical connections via wire and/or wireless communications to one or more remote computers, such as a remote computer 948. In various embodiments, one or more interactions described herein may occur via the networked environment. The remote computer 948 can be a workstation, a server computer, a router, a personal computer, portable computer, microprocessor-based entertainment appliance, a peer device or other common network node, and typically includes many or all of the elements described relative to the computer 902, although, for purposes of brevity, only a memory/storage device 950 is illustrated. The logical connections depicted include wire/wireless connectivity to a local area network (LAN) 952 and/or larger networks, for example, a wide area network (WAN) 954. Such LAN and WAN networking environments are commonplace in offices and companies, and facilitate enterprise-wide computer networks, such as intranets, all of which may connect to a global communications network, for example, the Internet.

When used in a LAN networking environment, the computer 902 is connected to the LAN 952 through a wire and/or wireless communication network interface or adaptor 956. The adaptor 956 can facilitate wire and/or wireless communications to the LAN 952, which may also include a wireless access point disposed thereon for communicating with the wireless functionality of the adaptor 956.

When used in a WAN networking environment, the computer 902 can include a modem 958, or is connected to a communications server on the WAN 954 or has other means for establishing communications over the WAN 954, such as by way of the Internet. The modem 958, which can be internal or external and a wire and/or wireless device, connects to the system bus 908 via the input device interface 942. In a networked environment, program modules depicted relative to the computer 902, or portions thereof, can be stored in the remote memory/storage device 950. It will be appreciated that the network connections shown are exemplary and other means of establishing a communications link between the computers can be used.

The computer 902 is operable to communicate with wire and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

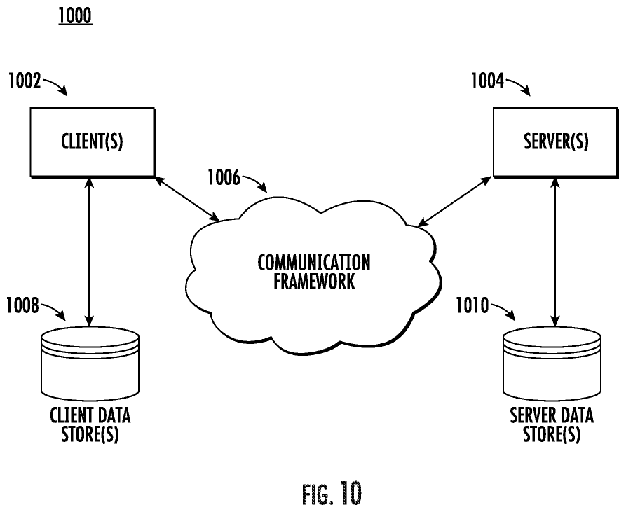

FIG. 10 illustrates a block diagram of an exemplary communications architecture 1000 that may be suitable for implementing various embodiments as previously described. The communications architecture 1000 includes various common communications elements, such as a transmitter, receiver, transceiver, radio, network interface, baseband processor, antenna, amplifiers, filters, power supplies, and so forth. The embodiments, however, are not limited to implementation by the communications architecture 1000.

As shown in FIG. 10, the communications architecture 1000 comprises includes one or more clients 1002 and servers 1004. In some embodiments, communications architecture may include or implement one or more portions of components, applications, and/or techniques described herein. The clients 1002 and the servers 1004 are operatively connected to one or more respective client data stores 1008 and server data stores 1010 that can be employed to store information local to the respective clients 1002 and servers 1004, such as cookies and/or associated contextual information. In various embodiments, any one of servers 1004 may implement one or more of logic flows or operations described herein, and storage medium 800 of FIG. 8 in conjunction with storage of data received from any one of clients 1002 on any of server data stores 1010. In one or more embodiments, one or more of client data store(s) 1008 or server data store(s) 1010 may include memory accessible to one or more portions of components, applications, and/or techniques described herein.

The clients 1002 and the servers 1004 may communicate information between each other using a communication framework 1006. The communications framework 1006 may implement any well-known communications techniques and protocols. The communications framework 1006 may be implemented as a packet-switched network (e.g., public networks such as the Internet, private networks such as an enterprise intranet, and so forth), a circuit-switched network (e.g., the public switched telephone network), or a combination of a packet-switched network and a circuit-switched network (with suitable gateways and translators).

The communications framework 1806 may implement various network interfaces arranged to accept, communicate, and connect to a communications network. A network interface may be regarded as a specialized form of an input output interface. Network interfaces may employ connection protocols including without limitation direct connect, Ethernet (e.g., thick, thin, twisted pair 10/100/1900 Base T, and the like), token ring, wireless network interfaces, cellular network interfaces, IEEE 802.11a-x network interfaces, IEEE 802.16 network interfaces, IEEE 802.20 network interfaces, and the like. Further, multiple network interfaces may be used to engage with various communications network types. For example, multiple network interfaces may be employed to allow for the communication over broadcast, multicast, and unicast networks. Should processing requirements dictate a greater amount speed and capacity, distributed network controller architectures may similarly be employed to pool, load balance, and otherwise increase the communicative bandwidth required by clients 1002 and the servers 1004. A communications network may be any one and the combination of wired and/or wireless networks including without limitation a direct interconnection, a secured custom connection, a private network (e.g., an enterprise intranet), a public network (e.g., the Internet), a Personal Area Network (PAN), a Local Area Network (LAN), a Metropolitan Area Network (MAN), an Operating Missions as Nodes on the Internet (OMNI), a Wide Area Network (WAN), a wireless network, a cellular network, and other communications networks.

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

In various embodiments, one or more of the aspects, techniques, and/or components described herein may be implemented in a practical application via one or more computing devices, and thereby provide additional and useful functionality to the one or more computing devices, resulting in more capable, better functioning, and improved computing devices. Further, one or more of the aspects, techniques, and/or components described herein may be utilized to improve the technical field of bioprocessing, filtration, tangential flow filtration, tangential flow depth filtration, and/or the like.

In several embodiments, components described herein may provide specific and particular manners of filtration processes in systems that include hollow fiber or TFDF cell retention elements. In many embodiments, one or more of the components described herein may be implemented as a set of rules that improve computer-related technology by allowing a function not previously performable by a computer that enables an improved technological result to be achieved. For example, the function allowed may include generating run parameters for filtration processes based on one or more user and/or administrative inputs including, without limitation, the solids content, pelleted cell volume, desired yield, retention factor and permeate throughput volume.

With general reference to notations and nomenclature used herein, one or more portions of the detailed description may be presented in terms of program procedures executed on a computer or network of computers. These procedural descriptions and representations are used by those skilled in the art to most effectively convey the substances of their work to others skilled in the art. A procedure is here, and generally, conceived to be a self-consistent sequence of operations leading to a desired result. These operations are those requiring physical manipulations of physical quantities. These quantities may take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in most cases, in any of the operations described herein that form part of one or more embodiments. Rather, these operations are machine operations. Useful machines for performing operations of various embodiments include general purpose digital computers as selectively activated or configured by a computer program stored within that is written in accordance with the teachings herein, and/or include apparatus specially constructed for the required purpose. Various embodiments also relate to apparatus or systems for performing these operations. These apparatuses may be specially constructed for the required purpose or may include a general-purpose computer. The required structure for a variety of these machines will be apparent from the description given.

1. (canceled)

2. (canceled)

3. A method of performing a filtration process, comprising:
receiving as user inputs: a process volume, a pellet cell volume, a solids cutoff, and optionally a filter retention value;
receiving as user or administrative inputs: a percent yield and a permeate throughput volume;
calculating run parameters using a control algorithm based on the user and administrative inputs;
beginning the filtration process run in concentration mode;
adding an aqueous solution using a diafiltration pump based on the run parameters that are calculated;
stopping diafiltration once a calculated number of diavolumes have been processed; and
ending the filtration process when the total permeate volume has reached the input permeate throughput volume.

4. The method of claim 3 wherein the control algorithm uses the percentage of a cell culture that is solid and the percentage of the cell culture which is liquid to calculate an expected product yield.

5. The method of claim 3 wherein the step of performing diafiltration occurs when the system reaches a predetermined percentage of solids.

6. The method of claim 3 wherein the step of stopping diafiltration further comprises stopping once a calculated percent yield is reached based on the number of diavolumes needed.

7-14. (canceled)

15. At least one non-transitory computer-readable medium comprising a set of instructions that, in response to being executed by a processor circuit, cause the processor circuit to:
receive as user inputs: a process volume, a pellet cell volume, a solids cutoff, and optionally a filter retention value;
receive as user or administrative inputs: a percent yield and a permeate throughput volume;
calculate run parameters using a control algorithm based on the user and administrative inputs;
begin the filtration process run in concentration mode;
add an aqueous solution using a diafiltration pump based on the run parameters that are calculated;
stop diafiltration once a calculated number of diavolumes have been processed; and
end the filtration process when the total permeate volume has reached the input permeate throughput volume.

16. The at least one non-transitory computer-readable medium of claim 15, wherein the control algorithm uses the percentage of a cell culture that is solid and the percentage of the cell culture which is liquid to calculate an expected product yield.

17. The at least one non-transitory computer-readable medium of claim 15, comprising instructions that, in response to being executed by the processor circuit cause the processor circuit to perform diafiltration when the system reaches a predetermined percentage of solids.

18. The at least one non-transitory computer-readable medium of claim 15, comprising instructions that, in response to being executed by the processor circuit cause the processor circuit to stop diafiltration when a calculated percent yield is reached based on the number of diavolumes needed.
